(19) United States
(12) Patent Application Publication
(10) Pub. No.: US 2018/0237602 A1
(43) Pub. Date: Aug. 23, 2018

(54) A transparent or semi-transparent nanostructured latex film for flexible and semi-transparent electronics for monitoring and manipulating cellular processes

(71) Applicant: ÅBO AKADEMI, Turku (FI)

(72) Inventors: Petri Ihalainen, Turku (FI); Anni Määttänen, Turku (FI); Sven Rosqvist, Turku (FI); Jouko Peltonen, Turku (FI); John Eriksson, Turku (FI); Erik Niemelä, Turku (FI); Arun Venu, Turku (FI); Pasi Kankaanpää, Turku (FI); Hend Abdelkader, Turku (FI); Fang Cheng, Turku (FI)

(21) Appl. No.: 15/755,136

(22) PCT Filed: Aug. 26, 2016
(86) PCT No.: PCT/FI2016/050590

§ 371 Date: Feb. 26, 2018

(30) Foreign Application Priority Data
Aug. 26, 2015 (FI) 20155608
Nov. 13, 2015 (FI) 20155840

(51) Int. Cl. C08J 5/18; C08J 7/12; G01N 33/483; C12M 1/34; C12M 1/32; C12N 5/00; C12M 1/00; C12M 1/12; D21H 19/20; G01N 27/30

(52) U.S. Cl. CPC C08J 5/18; C12N 2537/00; G01N 33/4833; C12M 41/46; C12M 23/12; C12N 5/0068; C12M 23/26; C12M 25/00; C12M 41/26; C12M 41/30; C12M 41/38; D21H 19/20; G01N 27/302; C08J 2355/02; C08J 2325/06; C12N 2535/00; C12N 2533/30; C08J 7/12

(57) ABSTRACT

Functionalized substrates with properties that can control cell-substrate interactions, induce cellular processes and decisions by means of passive and active control to enhance cell proliferation, cell migration and wound healing are provided. By including a sensing electrode on the substrate, it is possible to measure the metabolites of cells or follow the migration of additives like drug molecules in real-time. The present technology provides a new cell culture and imaging platform composed of a transparent and chemically and topographically customized latex film with electrodes that enable real-time measurement of e.g. pH and ion concentration in the cell medium as well as the metabolic states of the cells.

### FIELD OF INVENTION

The present invention is relating to a field of semi-transparent electronics for monitoring and manipulating cellular processes in real-time and to a field of latex films suitable for cell cultures.

### BACKGROUND ART

Traditionally, in vitro cell culture studies have been carried out using flat and clear glass or plastic well plates. Optical analysis of cell growth is commonly conducted using automatic high throughput microplate readers. A well-known issue of growing cells on flat and hard substrates is the differing manner in which they behave compared to the environments of living tissues. To be able to manipulate cell fate as well as measure metabolic states of cells would open up a new dimension of personalized disease treatment and screening.

A biocompatible and nanostructured latex blend has been proven to be a suitable substrate material for cell growth studies [1].

The influence of surface topography and in vivo mimicking of 3D features in cell cultures has been studied [2, 3, 4-6]. Textured surfaces have been fabricated by several methods, often by photolithography and etching [5]. In addition, nanoimprinting and different laser modification techniques have been also used for this purpose [7, 8]. Biodegradable thin films of poly-L-lactic acid [9] and chitosan [10] have been fabricated using soft lithographic techniques by applying the polymer solutions on the template surfaces and by peeling them off after solvent evaporation. Recently, a solvent treatment method for creating pores on flat polystyrene surfaces thereby gaining improved cell function mainly by enabling cells to form 3D aggregates has been described by De Rosa et al. [2]. Strain responsive wrinkling technique has been used by Choi et al. to create structured PDMS substrates [11]. Zhang et al. has used focused ion beam milling to create regularly patterned gold films with a wide palette of colors without employing any form of chemical modification [12]. Morariu et al. has described an electric field-induced sub-100-nm scale structure formation process using polymer bilayers [13].

### SUMMARY OF INVENTION

It is an aim of the present invention to further control the cells subjected to in vitro examination and to induce them to exhibit a more in vivo-like behavior.

It is another aim of the present invention to provide novel transparent or semi-transparent latex films for flexible and semi-transparent electronics, wherein said film is self-supporting or said film is on a transparent support.

It is a third aim of the present invention to provide a semi-transparent electronics assembly for monitoring and manipulating cellular processes in real-time.

The present invention is thus directed to a transparent or semi-transparent latex film for flexible and semi-transparent electronics, wherein said film is self-supporting or said film is on a transparent support.

The present invention is also directed to a semi-transparent electronics assembly for monitoring and manipulating cellular processes in real-time, comprising a latex film, which is self-supported or on a transparent support and semi-transparent electrodes for high-resolution microscopy imaging and electrical monitoring in real-time, with said electronics giving detailed information on one or several of cell morphology, growth and migration for multi-wells.

The present invention is further directed to a functionalized transparent or semi-transparent nanostructured latex film on a non-transparent support such as paper.

More specifically, the present invention is characterized by what is stated in the characterizing parts of the independent claims.

Considerable advantages are obtainable with the present invention. Thus, substrates with specific properties that can control cell-substrate interactions, induce cellular processes and decisions by means of passive and active control to enhance cell proliferation, cell migration and/or wound healing are provided.

By means of the present invention it is possible accurately to control the 3D structure (FIG. 1) and surface chemistry of the latex substrates as defined herein.

To be able to design surfaces that support, regulate, and stimulate biological processes is an approach, based on powerful material sciences, which has immense potential in applications of basic and applied research.

There is an immeasurable need within medicine, biotech and consumer market for inexpensive biocompatible materials that can be easily produced, even customized for specific needs. In many cases the possibility to sense and follow biological responses taking place within the employed materials would improve the understanding of the complicated cellular interactions with the surrounding environment. The present invention enables production of man-made materials with bio-supporting and bio-directing capabilities meeting such needs.

By including a sensing electrode on the latex substrate, it is possible, for example, to measure the metabolites of cells or follow the migration of additives like drug molecules in real-time. The present invention presents a new cell culture and imaging platform composed of a transparent and chemically and topographically customized latex film with electrodes that enable real-time measurement of e.g. pH and ion concentration in the cell medium as well as the metabolic states of the cells.

Next, embodiments will be examined in more detail.

### BRIEF DESCRIPTION OF THE DRAWINGS

FIG. 1: a) AFM topographical image (20 μm×20 μm) and b) line profile showing the dimensions of a self-supported latex film and the template grid used during its fabrication.

FIG. 2: a) A photograph and b) transmittance spectrum covering the visible wavelength area show the good transparency of the latex film and the evaporated gold electrodes. The numbers on the computer screen in the background are clearly visible through the gold film. A comparison of the topography prior to gold evaporation (c, d) and after gold evaporation on latex film (e, f) is seen in the AFM (5 μm×5 μm) images and the corresponding line profiles. The gold deposition can be seen as small grains of about 6 nm in height on top of the latex surface.

FIG. 3: A schematic presentation of different fabrication steps for latex films which for example are suitable for use as cell culture well plates and platforms.

FIG. 4: a) A photograph showing a liquid cell with gold electrodes on a latex film set up for impedance measurements. b) The long term stability of the electrode on the latex surface in cell media has been confirmed during impedance spectrometry. This is seen as a negligible change after the KCl electrolyte (black squares) is replaced by cell media (colored boxes). A much bigger change is expected when the electrodes are covered by cells. c) The capacitive response changes as the gold electrodes are functionalized with a self-assembled thiol layer indicating a successful surface modification.

FIG. 5. Height profiles of the latex surfaces with different blend ratios of HPY83 and DL920. This demonstrates that topography can be varied by varying blend ratio of latex components.

FIG. 6. a) A light microscope image of latex coated glass coverslip with high transparency, 10× magnification; b) A latex coated coverslip that have a electrode evaporated on the surface, transparency is around 85%, image taken with 10× magnification.

FIG. 7. Cell growth visible in the latex coated coverslips when Human Dermal Fibroblast (HDF) are grown on the surface. On the top, HDF cells grown on a non-coated glass cover slip, after 24 h (left) and 96 h (right). In the middle, HDF cells grown on a latex coated (HPY83:DL920—50:50) glass cover slip, after 24 h (left) and 96 h (right). In the bottom, HDF cells grown on a latex coated (HPY83:DL920—60:40) glass cover slip. Both transparent latex compositions show higher cell numbers compared to glass surface.

FIG. 8. Variations in HDF cell growth with a latex blend at different ratios (HPY83 to DL920), shown as cell growth relative to that observed on glass (normalized as 1.00). Nanostructured latex films dramatically affects cell growth.

FIGS. 9a, 9b & 9c. a) Water contact angles of the different surface blends of the HPY83:DL920 latex. The surface energy remains dominated by the hydrophilic component throughout the blends. b) Comparing cell growth on two latex blends with identical surface chemistry with the blends' root mean square roughness (S_{q}) normalized with the correlation length at 37% (Scl37)-S_{q}/Scl37—a peak value can be seen. Both surface parameters were extracted from 5 μm by 5 μm AFM images. c) Comparing cell growth on two latex blends with identical surface chemistry with the blends' Surface Area Ratio (area of 3D surface per area 2D (flat) surface). The nano scale roughness affects cell growth. The surface parameters were extracted from 5 μm by 5 μm AFM images.

FIG. 10. Human dermal fibroblasts (HDF, top row) or human cervical cancer cells (HeLa, bottom row) were seeded and administered doxorubicin (DOX) on either a standard 24 well plates (“plate reader”) or on our latex coated paper based screening assay (“paper pipeline”). Both the HDF and HeLa cells were subjected to an increasing amount of doxorubicin from 0.05 μg/ml up to 5 μg/ml. After 24 hours, each well of both assays were stained with crystal violet dye in order to detect the viable cells. The multi-well plate was analyzed using a standard plate reader using a 570 nm absorbance measurement and the wells of the paper pipeline was first scanned with a nor trial office scanner using an RGB (Red Green Blue) color system and then quantified by our in-house made analytical software. The raw data from both methods were then normalized to the highest value in order to get the 100% viable control sample. The results shows that our paper based screening assay is faster, more sensitive and affordable compared to the standard plate reader when using crystal violet as the viability assay.

Scheme 1 is a schematic illustration of hierarchically structured latex based semi-transparent electronics for monitoring and manipulating cellular processes in real-time.

### DESCRIPTION OF EMBODIMENTS

Cell signaling governs the fate of all cells. In the present invention, materials with specific properties have been developed and an understanding gained how cell-substrate interactions control cellular processes and decisions by means of passive and active control to enhance tissue regeneration.

Wound healing is an appropriate model for studying a regenerative procedure which is formed by three partially overlapping phases: inflammation, cell proliferation, and tissue remodeling. Results obtained from this model can be applied to other areas of regeneration. Surfaces with different physicochemical properties and biofunctionalities affect the cellular state as shown herein.

Printing and imaging technologies can also be employed.

As discussed above, functionalized substrates with properties that can control cell-substrate interactions, induce cellular processes and decisions by means of passive and active control to enhance cell proliferation, cell migration and/or wound healing are provided.

By including a sensing electrode on the substrate, it is possible, for example, to measure the metabolites of cells or follow the migration of additives like drug molecules in real-time.

As a substrate suitable for supporting cell cultures and compatible for flexible and semi-transparent electronics, the present invention provides a transparent or semi-transparent latex film, wherein said film is self-supporting or said film is on a transparent support.

Thus, typically, the substrate comprises a structure which is transparent or semi-transparent and extends preferably along a plane, such that it allows for transmission of light, in particular light in the visible range, through the structure, for example at an angle of 45 to 135°, in particular 60 to 120°, for example about 90°, against the plane along which the substrate extends.

The transparent support can preferably be made of glass or polymer material, such as a thermoplastic material. Examples of transparent supports are glass coverslips and microwell plates such as microtiter plates.

Preferably, the film is transparent or semi-transparent with the transmission of light in visible range being over 50%, more preferably in the range of 70-90%.

The terms “transparent” and “semi-transparent” refer herein to the field of optics so that transparency is understood as the physical property of allowing visible light to pass through the material without being scattered.

The transmission through the material, as discussed herein, is for example measured at an angle of 45 to 135°, in particular 60 to 120°, for example about 90°, against the plane along which the substrate extends.

The term “roll-to-roll processing” refers herein to the process of creating electronic devices on a roll of flexible plastic or metal foil. It can refer to any process of applying coatings or printing by starting with a roll of a flexible material and re-reeling after the process to create an output roll.

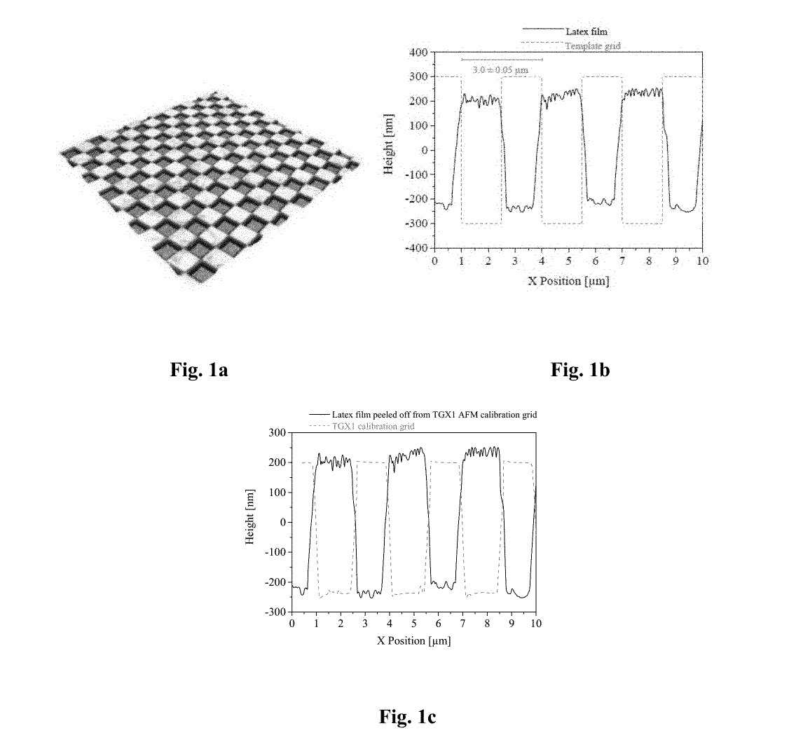

In one major embodiment of the invention, the latex film comprises a nanostructured surface having a hierarchical morphology. An example of the hierarchical morphology is shown in FIG. 1a. Latex blends used for preparing said films preferably comprise styrene and/or butadiene groups. Thickness of the present latex film varies preferably in the range of 3-40 nm, more preferably 5-30 nm. Said nanostructured surface can be formed by a heat treatment, e.g. by sintering the latex film with an IR lamp.

Preferably, said latex film comprises a blend of two latexes (i.e. “hard” and “soft” latexes). More preferably, said two latexes comprise polymers selected from the group consisting of: styrene, acrylonitrile, butadiene (i.e. 1,3-butadiene) and copolymers thereof. Most preferably, said two latexes are polystyrene and styrene butadiene acrylonitrile copolymer. Said two latexes are preferably mixed about 40:60, 50:50 or 60:40. Preferable particle size for polystyrene is 100-200 nm providing barrier properties, mechanical strength and integrity for the film.

The present technology thus provides a new cell culture and imaging platform composed of a transparent and chemically and topographically customized latex film with electrodes that enable real-time measurement of e.g. pH and ion concentration in the cell medium as well as the metabolic states of the cells.

In a further embodiment, a semi-transparent or transparent electronics assembly is provided for monitoring and manipulating cellular processes in real-time. In particular the assembly comprises a hierarchically structured latex. Such an assembly is formed by
a transparent substrate with a deposited latex layer having a predetermined structure; and
semi-transparent or transparent electrodes for high-resolution microscopy imaging.

Preferably, the electrodes allow for electrical monitoring for example in real-time.

Preferably, the electronics assembly gives detailed information on one or several of the following features: cell morphology, growth and migration for multi-wells and other corresponding platforms.

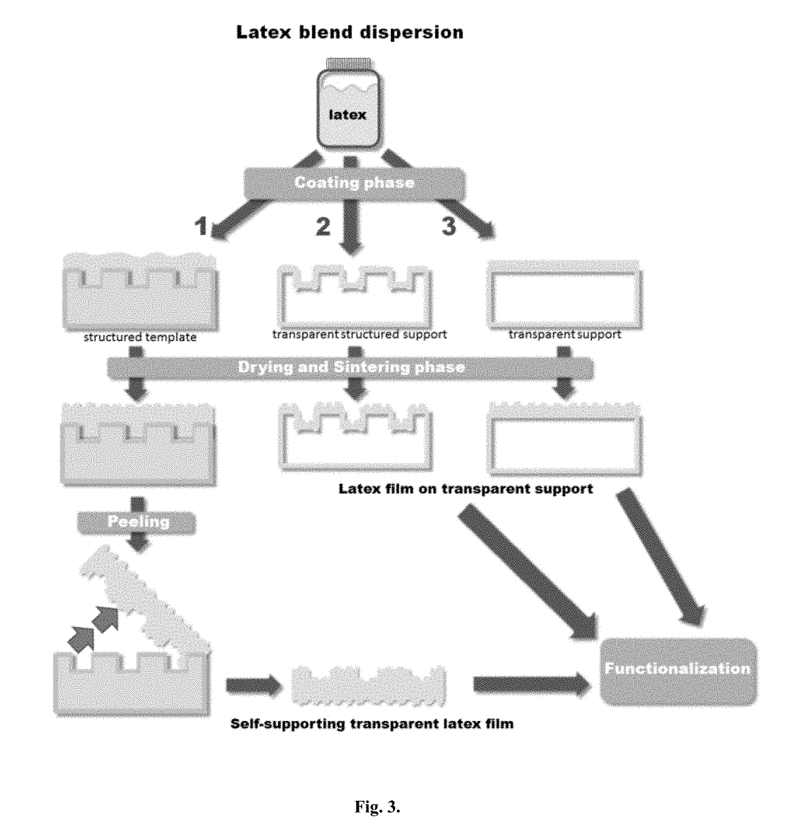

A schematic presentation of different fabrication steps for latex films is shown in FIG. 3. The latex comprises or consists of a synthetic or naturally occurring stable aqueous dispersion or emulsion of polymer particles, preferentially containing styrene and/or butadiene groups. The blend used is typically a mixture of two or more of aforementioned emulsions or dispersions.

The fabrication comprises four steps:
the coating phase,
the drying and sintering phase,
the peeling phase and
the functionalization phase.

The peeling phase is only necessary for the fabrication of self-supporting films, while the functionalization phase only applies if latex surfaces are desired to carry a surface functionalization. In the image three example lines are shown.

In the first line (1), latex is coated on the surface of a structured template, dried and sintered to obtain a desired surface, and finally peeled off to become a self-supporting latex film substrate.

In the second line (2), a latex coating is spread on a structured supporting substrate, and dried and sintered, to enable the design of a hierarchically structured surface.

Similarly, in the third line (3), latex is directly coated on a transparent supporting substrate without structure.

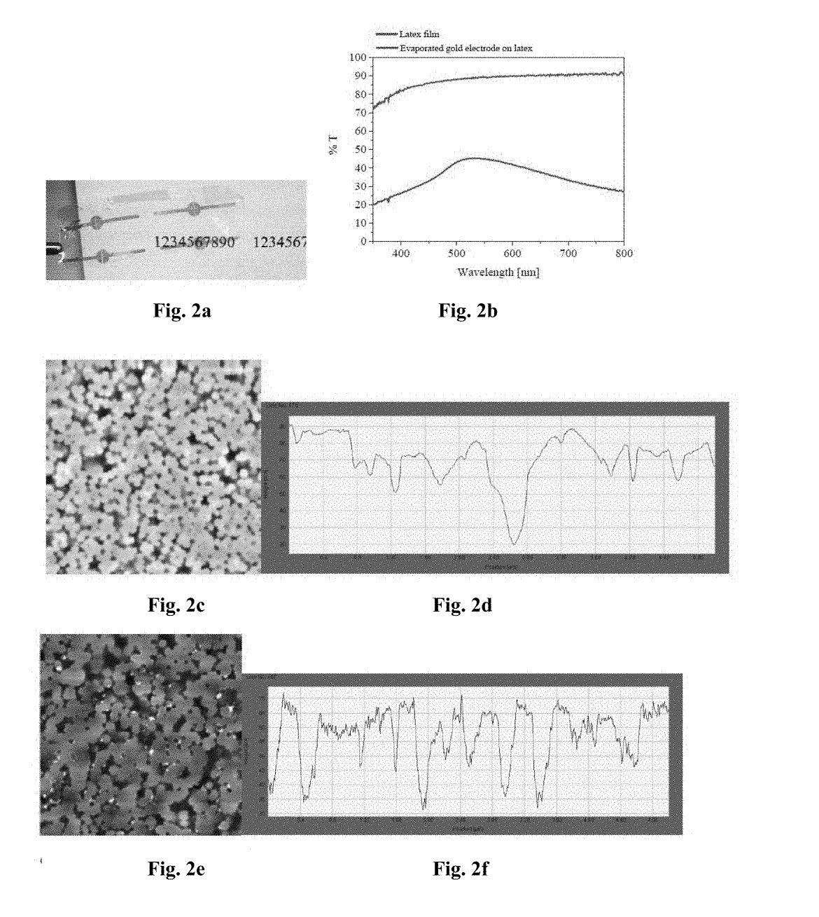

Different template materials can be used for creating various 3D structures for the latex substrates, in particular so as to form well plates for in vitro cell culture studies. Different latex blends and heat-treatments give rise to different topography and surface chemistry. The highly transparent latex films can be self-supported as for example in FIGS. 1 and 2a or then supported by for instance by glass cover slips (FIG. 3a) or paper (FIGS. 3b to 3d). A similar bimodal nanostructured surface topography is obtained for self-supported, paper and glass supported substrates.

Electrically and electrochemically active semi-transparent layers for electric modulation and sensing can be deposited on the latex. For example, ultra-thin and conductive gold electrodes (UTGE) with 50% transmission Ultra-thin and conductive gold electrodes (UTGE) can be evaporated onto the latex surface (FIG. 2a). A preferred alternative is a conductive semitransparent or transparent polymer such as PEDOT:PSS.

UTGEs with nominal thickness of 20 nm were fabricated using physical vapor deposition with resistive heating and a shadow mask for patterning. The evaporation was done under high vacuum (10-6 mbar) using a heated aluminum-coated tungsten basket. A deposition monitor (XTM/2, Inficon) was used for gravimetric determination of the amount of evaporated gold on the film surface. With a nominal thickness of 20 nm, conductive UTGF electrodes (resistivity: 2.6×10−6 Ω cm) with grain thickness of about 6 nm were obtained.

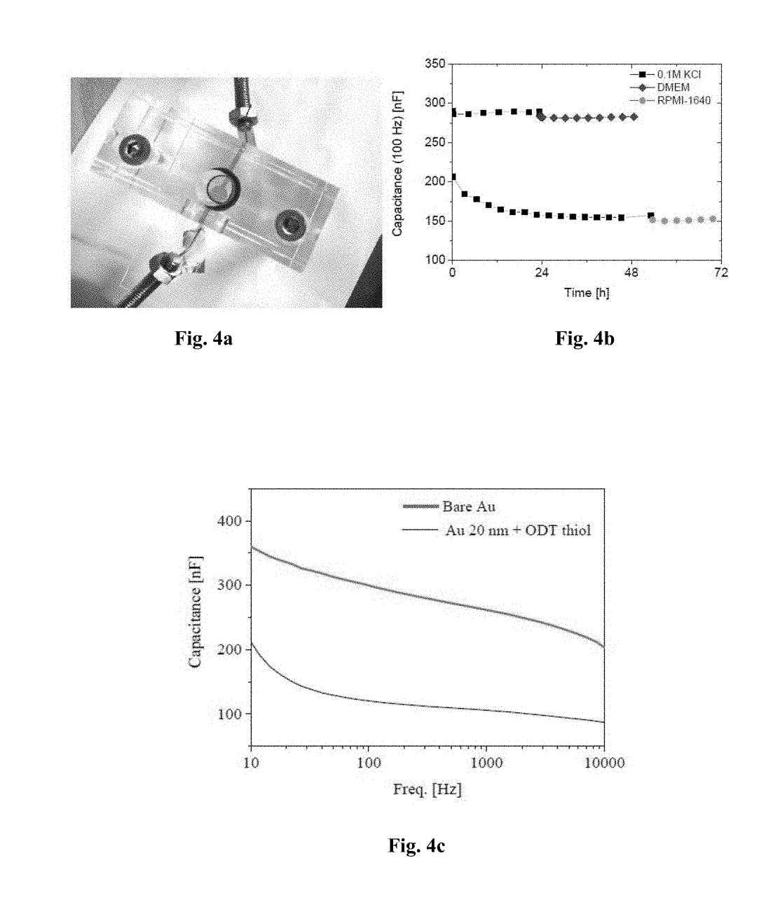

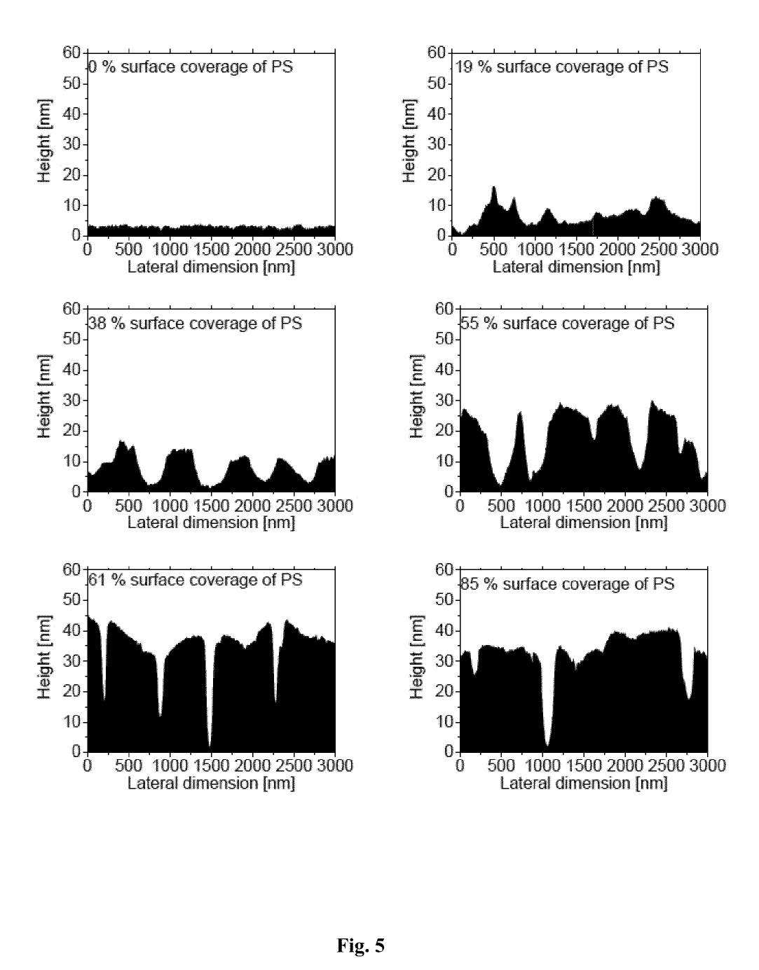

The latex and electrode surfaces can be further or alternatively functionalized e.g. by printed biomolecule films or self-assembled thiol monolayers (FIG. 4). Impedimetric studies confirmed a good long term stability of the electrodes in cell media, which is necessary for applications in the field of cell growth, migration and proliferation where the time span of various processes can be several days (FIG. 4b). Preferred biomolecules for functionalization also include active pharmaceutical ingredients (API) or other chemical compounds, such as toxic chemicals, having an effect on cell growth or activity.

The bare gold electrodes can also be used as such for measuring the concentration of electroactive analytes using cyclic voltammetry when an appropriate reference electrode is used. As an example the platforms can be used to determine the concentration of active medicinal components as demonstrated with caffeic acid in the Experimental Section below.

The electrodes can also be modified for instance by electro polymerizing a conductive polymer layer that allows a continuous monitoring of the pH or concentration of glucose or other cell metabolites in the cultivation area during cell growth. By using ion selective membranes on the electrode the concentration of different ions, e.g. potassium [K+], can be analyzed.

The transparency of both the latex substrate and the thin electrodes (FIG. 2) enables simultaneous high resolution optical imaging and electrical measurements which opens up direct correlation of variable parameters to cell growth, and further to e.g. wound healing. Specific advantages of electrical methods are the ability to detect low concentrations of biological analytes and the label-free analysis techniques.

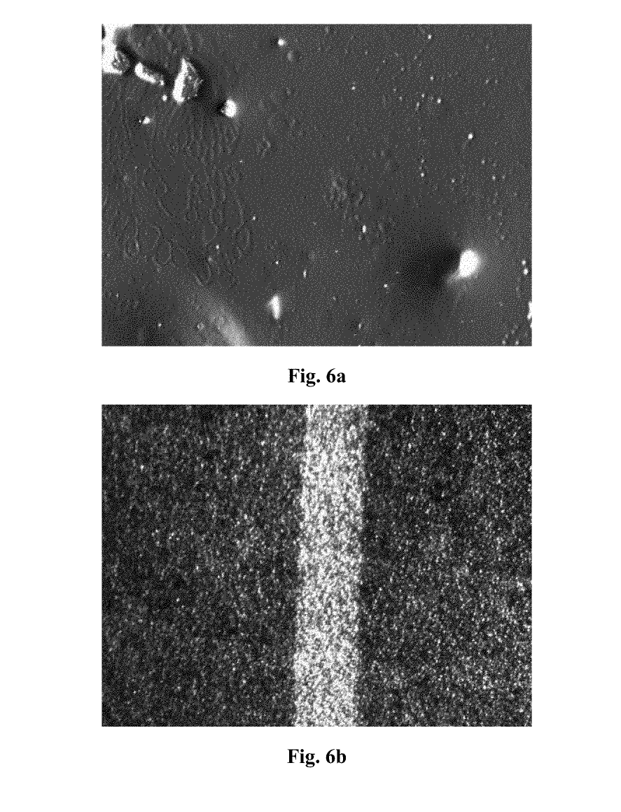

One typical platform is a transparent modified glass coverslip substrate with a latex-based structured surface topography, tailored surface chemistry and semi-transparent electrodes for high-resolution microscopy imaging and electrical monitoring in real-time, giving detailed information on cell morphology, growth and migration (FIG. 6).

The materials can be designed to control cell growth or induce cell migration in a desired manner. The electrodes open up the possibility to control cell fates by electrical stimulus, controlled drug release and to simultaneously measure pH and metabolites in the cell medium as well as glucose levels during optical imaging of cell growth so that a new dimension of cell culture measurement can be born (FIG. 6; Scheme 1).

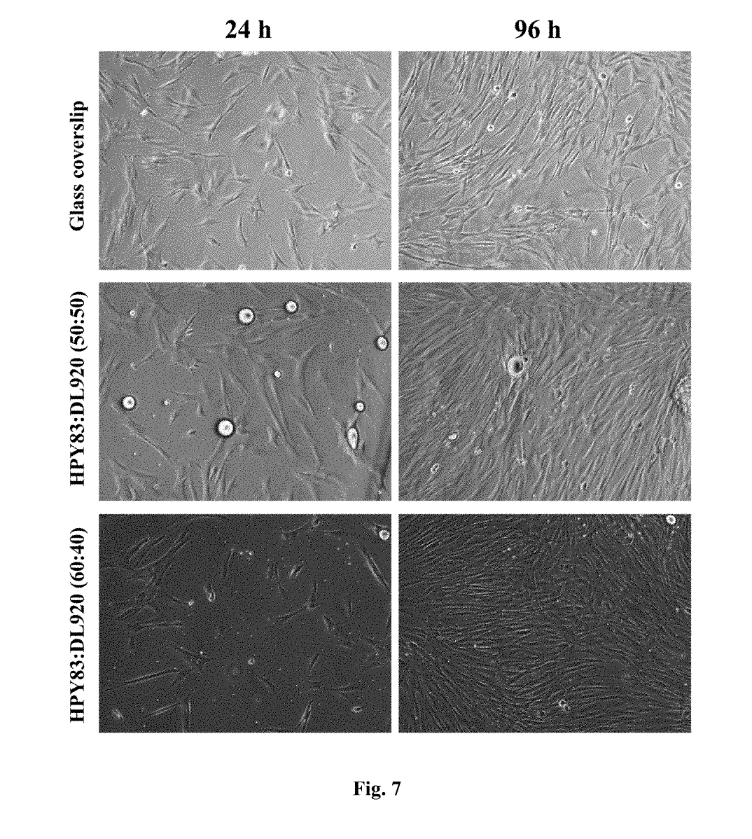

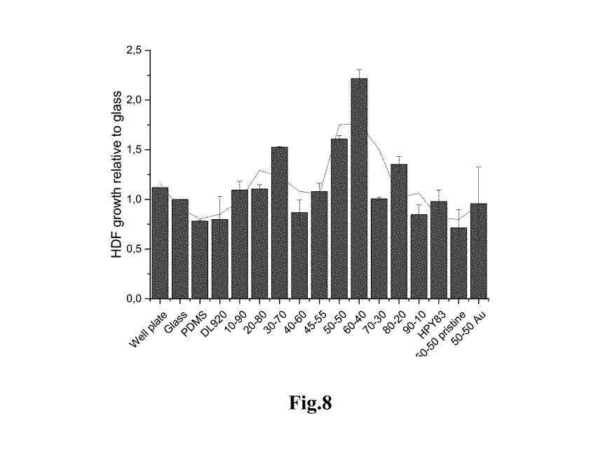

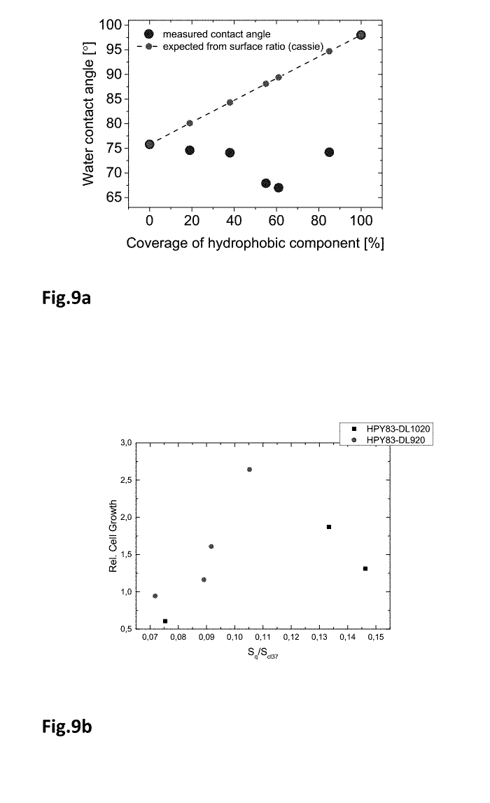

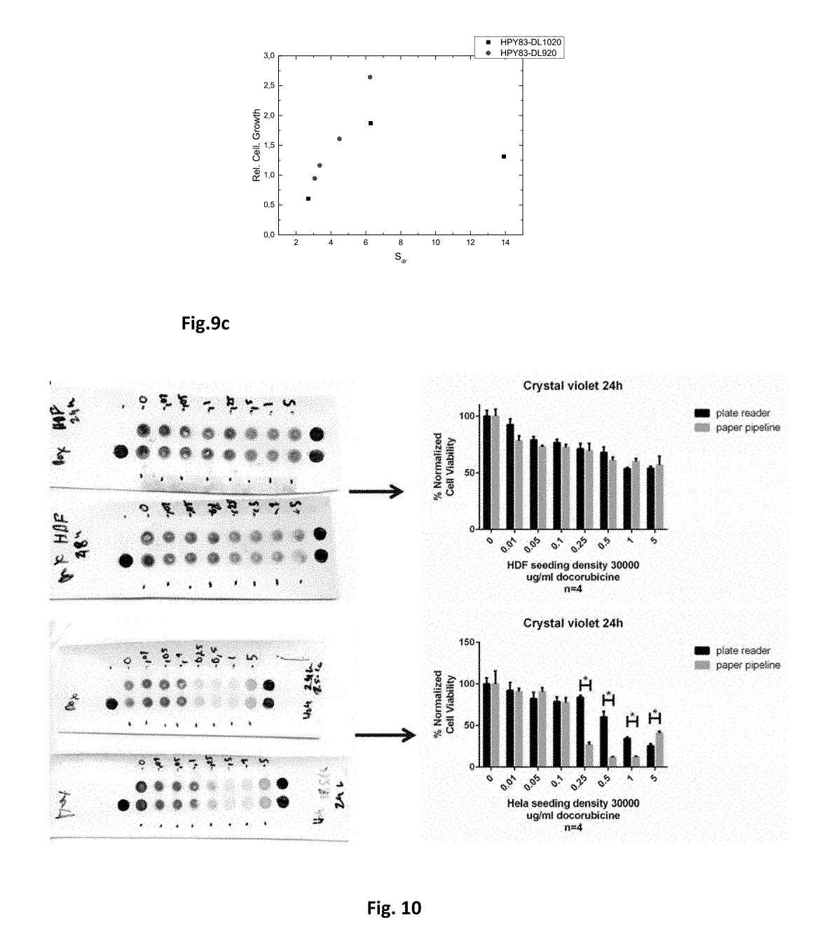

As seen in FIG. 7 an clear increase of cell growth is visible after 3 days of incubation in the latex coated coverslips when Human Dermal Fibroblast (HDF) are grown on the surface. This increased cell growth could be utilized when a rapid cell division is desired, such as in the case of wound healing.

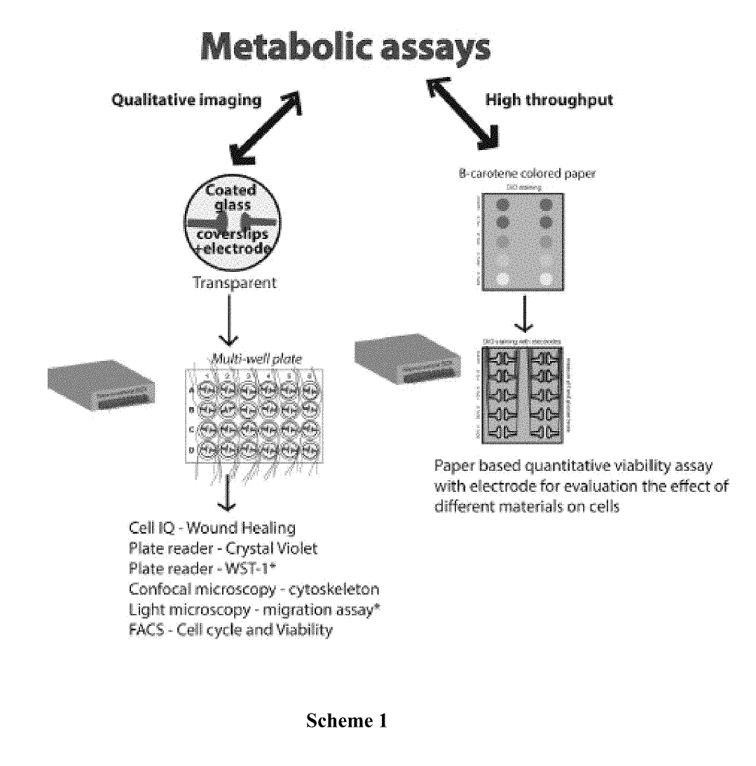

Another platform type is paper or similar natural fibre based material combined or supported with a structured and functionalized latex-film that can be mass produced at low cost. This version is suitable for fast and robust quantification of cell growth on materials of interests, e.g. as a screening platform (see FIG. 10). The material of interest or the active pharmaceutical ingredients (API) could either be deposited (eg. coated, printed etc.) directly on the paper or then added to the medium. In short the paper could be colored with a non-toxic dye (e.g. B-carotene, food colorants) to discriminate the region of interest (ROI); the ROI would then be an un-dyed circular area where the cells would grow (Scheme 1). Alternatively, the ROI could be limited by a wetting controlling material (e.g. hydrophobic polydimethylsiloxane (PDMS) or waxes, etc.).

The latex-film can be subjected to validation which is being analyzed by coloring the viable/non-viable cells with a color; such as DiO/DiI (green/red fluorescence), crystal violet (blue dye) and/or other colors belonging to the group of lipophilic fluorescent stains for labeling cell membranes and other hydrophobic structures.

The intensity of the color can be measured with a colorimetric analysis of the dye that would correlate to cell amount on the material of interest with or without administered API.

Typically, the color intensity is measured with a scanner and then quantified with an analytical tool capable of giving an estimate of the cell viability of the material of interest compared to an known control surface where cells grows on.

Scheme 1. Schematic illustration of hierarchically structured latex based semi-transparent electronics for monitoring and manipulating cellular processes in real-time:

Transparent modified glass coverslip substrate with a latex-based structured surface topography, tailored surface chemistry and semi-transparent electrodes for high-resolution microscopy imaging and electrical monitoring in real-time, giving detailed information on cell morphology, growth and migration for multi-wells. The materials can be designed to control cell growth or induce cell migration in a desired manner. The electrodes open up the possibility to control cell fates by electrical stimulus, controlled drug release and to simultaneously measure pH and metabolites in the cell medium for real-time metabolic measurements.

In the following Experimental Section, latex films according to the present technology were used as substrates for evaporated ultrathin and semi-transparent gold electrodes with nominal thicknesses of 10 nm and 20 nm. Optical properties and topography of the samples were characterized using UV-vis spectroscopy and Atomic Force Microscopy (AFM) measurements, respectively. Electrochemical impedance spectroscopy (EIS) measurements were carried out for a number of days to investigate the long term stability of the electrodes. The effect of 1-octadecanethiol (ODT) and HS(CH2)11OH (MuOH) thiolation and protein (human serum albumin, HSA) adsorption on the impedance and capacitance was studied. A typical ˜10% decrease of capacitance at 100 Hz was observed after immobilization of 1 mg/mL HSA on the bare and ODT functionalized gold electrodes in still conditions. The corresponding change of capacitance on the hydrophilic MuOH functionalized electrode was negligible. The performance of the electrodes was tested also under flow conditions with EIS measurements. In addition, cyclic voltammetry (CV) measurements were carried out to determine active medicinal components, i.e., caffeic acid with interesting biological activities and poorly water soluble anti-inflammatory drug, piroxicam.

### EXPERIMENTAL SECTION

1. Materials and Methods

1.1 Template Substrates

Four different AFM calibration grids (models: TGG1, TGZ2, TGT1 and TGX1, NT-MDT, Russia), microscope glass slides (Menzel-Gläser, Thermo scientific, Germany), Polydimethylsiloxane (PDMS) [36] (Wacker, Germany) and a multilayer curtain coated paper [38] were used as model template substrates from which the latex coatings were peeled off.

1.2 Coating Material

The two component coating latex blend with a weight ratio of 1:1 was prepared by mixing aqueous dispersions of polystyrene particles (HPY83; average particle size=140 nm, Tg=105° C., wt. %=48.0, DOW) and styrene butadiene acrylonitrile copolymer (DL920; average particle size=140 nm, Tg=8-10° C., wt. %=49.5-50.5, DOW).

1.3 Latex Film Fabrication

Different film fabrication methods were used, for example rod coating was applied on the paper and glass substrates and drop-casting was used on the calibration grids. After the films appeared dry, they were sintered using an IR lamp (IRT systems, Hedson Technologies AB, Sweden) for 45-60 s in order to fuse the particles together. The samples were immersed in water and washed in an ultrasound bath (FinnSonic m08) for 10 s and then the latex films were peeled off from the template substrates. The fidelity of the replication technique greatly depends on the properties of the template materials. For example peeling of a thin latex film from a more porous precipitated calcium carbonate (PCC) coated paper substrate was not feasible. On the other hand, the low surface energy, durability, flexibility and low adhesive force [16] of polydimethylsiloxane (PDMS)—based templates make them ideal template materials. Thickness of the latex film also has an influence, i.e., thicker latex films are generally easier to peel off from the templates, but their trying time is long and transparency lower. Naturally the shape of the templates also somewhat influences the fidelity of the peeling process. For example the latex film was easier to peel off from the TGZ2 grid (with vertical and horizontal surface features) compared to TGX1 grid with chessboard-like array of square pillars with sharp undercut edges. With a low coating amount the IR treatment reached throughout the whole coating thickness creating the characteristic nanopatterned structure within the higher hierarchical pattern. In case of thicker coating amounts, an additional IR treatment could be performed after the peeling process to obtain a typical heat-treated surface structure also on the bottom side.

1.4 Fabrication and Functionalization of Ultrathin Gold Film Electrodes

The ultrathin gold films (UTGF) with nominal thicknesses of 10 nm and 20 nm were fabricated on the self-supported latex films using physical vapour deposition (PVD) with resistive heating. The film was attached on the shadow mask that was used for patterning. The gap between the evaporated gold electrodes was ˜190 μm and the width of the electrodes 5 mm. The dimensions of the contacts were 1 mm×12 mm. The evaporation was done under high vacuum 2-5×10−6 mbar during two separate runs using a heated aluminium-coated tungsten basket. The evaporation rate was set to 1 Å/s. A deposition monitor (XTM/2, Inficon) was used for gravimetric determination of the amount of evaporated gold on the film surface. The topographical characterization and electrochemical application of the UTGF electrodes on paper-supported latex coatings have been previously described elsewhere [17]. Briefly, a nominal thickness of 10 nm yielded UTGF electrodes with semiconducting (n-type) characteristics and polycrystalline grain structure with grain thickness of about 2 nm. Respectively, a nominal thickness of 20 nm yielded conductive UTGF electrodes (resistivity: 2.6×10−6 Ω cm) with grain thickness of about 6 nm. Similar characteristics were observed also for the UTGF electrodes on the self-supported latex film.

Functionalization of the UTGF electrodes with a self-assembled monolayers (SAMs) were carried out with a hydrophobic 1-octadecanethiol (ODT, Fluka Chemika) in ethanol and with a hydrophilic HS(CH2)11OH (MuOH, Sigma-Aldrich) in water. Before thiolation, the evaporated UTGF electrodes were cleaned with plasma (air) flow (PDC-326, Harrick) for 2 min and rinsed or immersed in absolute ethanol. The plasma treated self-supported latex films with UTGFs were placed on a microscope glass support and sealed with a silicone ring in a custom-built liquid flow cell (FIAlab Instruments, Inc., USA) (Appendix, A1) and exposed to the thiol solution (ODT: 500 μL, 5 mM/MuOH: 500 μL, 446 μM) for 24 h at room temperature under a cap. After the SAM formation, the ODT-functionalized electrodes were rinsed with absolute ethanol and 0.1 M KCl and the MuOH-functionalized electrodes with water and 0.1 M KCl solution. The HSA protein adsorption studies were conducted using 0.1 M KCl as the supporting electrolyte.

1.5 Characterization

Transmission UV-vis spectroscopy measurements were carried out through a black board mask with a 5 mm×5 mm hole using a Perkin-Elmer Lambda 900 with an integrating sphere setup.

Electrical Impedance spectroscopy (EIS) measurements were performed using a portable electrochemical interface and impedance analyzer (CompactStat, Ivium Technologies, The Netherlands). The experiments were carried out with a two electrode setup for keeping the electrode construction planar and simple. An aluminum foil was placed on top of the ultrathin gold electrode contacts before thin metal probes were pressed on the contacts connecting the gold electrodes to the CE and WE cables of the instrument. The electrolyte solution was applied on top of the electrodes using a liquid cell. A capacitance vs. potential plot for the gold electrodes with 10 nm and 20 nm nominal thicknesses was first measured in 0.1 M KCl to determine the point of zero charge (E˜0 V). The impedance measurements throughout the work were recorded at a constant dc-potential (0 V) and with an applied sinusoidal excitation signal of 10 mV at a frequency range of 10000 Hz-10 Hz. In the flow measurements the solutions with a total volume of at least 5 mL were circulated with a flow rate of 23 μL/s using a peristaltic pump (101U/R Watson Marlow, England).

CV measurements were carried out using the same CompactStat and liquid cell setup. The electrode system consisted of an gold working electrode (WE), an gold counter electrode (CE) and a conventional Ag/AgCl (3M KCl) (Metrohm) reference electrode. The electrodes were not placed in the middle of the liquid cell but slightly off so that the area of the WE (˜7.3 mm2) was smaller than the area of the CE. A scan rate of 25 mV/s was used and the potential was cycled between −0.2 V and +0.8 V in case of caffeic acid (3-(3,4-dihydroxyphenyl)-2-propenoic acid) solution and between 0 V and +0.8 V in case of piroxicam (4-hydroxy-2-methyl-N-(2-pyridyl)-2H-1,2-benzothiazine-3-carboxamide-1,1-dioxide) solution. 0.1 M KCl in water was directly used as the supporting electrolyte.

An NTEGRA Prima (NT-MDT, Russia) atomic force microscope (AFM) was used for analyzing the surface topography of the peeled latex films. The images were scanned in air operating with intermittent-contact mode at the repulsive regime using rectangular cantilevers (NSG10 NT-MDT, Russia) with a 0.3 Hz scan rate at ambient conditions (T=27±2° C., Relative humidity, RH=44±3%). The images were processed and analyzed using the SPIP (Scanning Probe Image Processor, Image Metrology, Denmark) software. Contact angle measurements were carried out with a CAM200 contact angle goniometer (KSV Instruments Ltd.) at ambient conditions (T=29.8° C., RH=38.4%). Small 2 μL sized water (Millipore) droplets were placed on the samples and the contact angle values were recorded as a function of time.

2. Results and Discussion

2.1 Preparation and Topographical Characterization of Hierarchically Structured Self-Supported Films and Semi-Transparent Electronics

Different kinds of template substrates were used for the preparation of the self-supported latex films depending on the hierarchical structure desired. For example, sub-nanometer and nanometer scale features can be prepared by rod-coating the latex blend dispersion on a pigment coated paper substrate. After an IR treatment a distinct nanostructured topography with bimodal height distribution and random distribution, depending on the ratio of soft and hard components in the latex blend [19] was obtained. It is notable that the top-side structure of the self-supported film remained unchanged compared to the structure of the coating still being attached to the supporting substrate indicating that the peeling-off process did not cause any apparent changes or defects on the surface structure of the latex film (with a thickness of approximately 5.1 μm).

Higher hierarchical ordering can be achieved by applying the latex coating on substrates with lithographically pre-patterned structures. Here, we used AFM calibration grids due to their very precise sub-micron or micron periodic structure. After coating, IR sintering and agitation treatment, the latex film was peeled off. The periodic structures on both the latex film and the calibration grid appear as rainbow-like iridescent colors. The colors are created by structural coloration [18] and thus appear only on the effective 3 mm×3 mm central square of the 5 mm×5 mm TGZ2 chip. Comparison of vertical and lateral dimensions of the surface features in the AFM line profiles to the dimensions of the AFM calibration grid gratings show that a negative replica of the calibration grid structure was very accurately produced.

2.2 Optical Characterization

Optical transparency of the self-supported latex films was determined by UV/vis spectrophotometer in transmission mode. About 80% optical transmission in the visible light region (400-700 nm) was achieved with the self-supported films that were peeled off from a paper substrate and wetted with water or soaked with linseed oil from the back side. About 10% less light was transmitted when the films were in dry state. This change was clearly seen also by naked eye. To create a typical bimodal surface on both sides of the peeled latex films, a glass slide was used as the template. Thereby also the optical transparency was enhanced to approximately 90%.

The optical transparency of the self-supported latex films decreased to around 45-50% after the deposition of UTGF electrodes. For comparison, the optical transparency of an ITO top electrode (processed at low temperature) used in solar cells has an average transmittance of above 85% [20].

The UTGFs had a typical polycrystalline grain morphology commonly observed for vapor-deposited UTGFs [17]. The average grain height in the UTGFs with a nominal thickness of 10 nm and 20 nm was 2.5±0.5 and 6.2±0.3 nm, respectively. These correspond to the height values previously obtained for UTGFs on paper-supported latex coating [17]. The lack of a clear dip in transmission after ˜500 nm typically observed for discontinuous UTGFs due to localized surface plasmon resonance absorption [21] indicates that UTGFs on self-supporting latex film are quite continuous. UTGFs on paper-supported latex coatings have been shown to form a continuous, interconnected island network on the surface even with nominal thickness of 10 nm [17]. This seems to be true also here and explains the high conductivity of UTGF with nominal thickness of 20 nm [17]. The thicker UTGF showed a pronounced decrease in optical transmission at longer wavelengths whereas the transmission of the thinner UTGF remained quite stable. This trend follows that shown for ideal UTGFs (i.e. consisting of a single Au layer with homogeneous density) by theoretical calculations [19]. Theoretical transmission curves calculated by the transfer-matrix method using the bulk dielectric function of gold predict a faster drop of the optical transmission in VIS/NIR region as a function of film thickness. The resistance (R) of the UTGF evaporated on the latex film peeled off from the TGZ2 template surface was measured with a Fluke 73 III multimeter using two probes at a distance of 4 mm from each other. Almost equal R values were measured when the probes were placed in parallel with the lines (9.7Ω) and across the lines (11.4Ω). This further demonstrates the good continuity of the evaporated gold films even on structured surfaces.

2.3 Electrochemical Characterization

Impedimetric measurements have been carried out with paper-based printed and evaporated gold electrodes previously [17, 22-24] in steady state. Here the EIS studies were carried out with the transparent self-supported nanostructured latex versions for extended time periods as a good long term stability of the UTGF electrodes is necessary e.g. in the field of cell growth, migration and proliferation where the time span of various processes can be several days. Good barrier properties are important for obtaining stable readings in liquid medium. One benefit related to the use of the self-supported latex films is that in case of a small pinhole or defect in the latex film (or substrate with inadequate barrier properties e.g. pristine latex coating) there is no supporting base paper substrate that would suck the liquid or solution which would cause e.g. unwanted concentration changes. The capacitance of the ODT-functionalized electrodes remained extremely constant at 133±2 nF for several hours after the initial stabilization. The obtained capacitance decrease from 202 nF was approximately 34%.

CV measurements were carried out with two pharmaceutically interesting model compounds, i.e., caffeic acid and piroxicam. 0.1 M KCl in water was directly used as the supporting electrolyte without any optimization to lower the oxidation potential of the compounds e.g. by changing the solution pH or the electrolyte and its concentration [25]. The profiles of the cyclic voltammograms measured with the highest caffeic acid concentration are quite characteristic for caffeic acid sample showing one anodic peak at 505 mV and one cathodic peak at 280 mV [25]. Piroxicam on the other hand is voltammetrically oxidizable and showed only the oxidation peak [26].

The following clauses are characteristic of further embodiments of the present invention:

1. Semi-transparent electronics assembly for monitoring and manipulating cellular processes in real-time, comprising a hierarchically structured latex formed by
a transparent modified glass coverslip substrate with a latex-based structured surface topography,
tailored surface chemistry and
semi-transparent electrodes for high-resolution microscopy imaging and electrical monitoring in real-time,

said electronics giving detailed information on one of several of cell morphology, growth and migration for multi-wells.

2. The assembly according to clause 1, wherein the materials are designed to control cell growth or induce cell migration in a desired manner.

3. The assembly according to clause 1 or 2, wherein the electrodes allow for controlling cell status by electrical stimulus, controlled drug release while simultaneously measuring pH and metabolites in the cell medium for real-time metabolic measurements.

4. Paper supported functionalized latex-film which preferably can be mass produced at low cost, useful for fast and robust quantification of cell growth on materials of interests such as active pharmaceutical ingredient (API), comprising a paper preferably colored with a non-toxic dye such as β-carotene to discriminate a region of interest (ROI);

the ROI being formed by a circle without the dye; and inside this circle the material of interest being printed on in order to validate if cells thrive on the surface or not.

5. Use of the latex-film according to clause 4, wherein validation being analyzed by coloring the viable cells with a color; such as DiO or crystal violet, which intensity would be measured with a colorimetric analysis of the dye that would correlate to cell amount on the material of interest such as active pharmaceutical ingredient (API).

6. The use according to clause 5, wherein color intensity is measured with a scanner and then quantified with an analytical tool capable of giving an estimate of the cell viability of the material of interest compared to an known control surface where cells grows on.

### CITATION LIST

### Non-Patent literature

1. Helka Juvonen, Anni Määttänen, Patrick Laurén, Petri Ihalainen, Arto Urtti, Marjo Yliperttula, Jouko Peltonen, Biocompatibility of printed paper-based arrays for 2-D cell cultures, Acta Biomaterialia, 9, 2013, 6704-6710.

2. M. E. DeRosa, Y. Hong, R. A. Faris, H. Rao, Microtextured polystyrene surfaces for three-dimensional cell culture made by a simple solvent treatment method, J. Appl. Polym. Sci. 131 (2014) 40181.

3. J. Lee, M. J. Cuddihy, N. A. Kotov, Three-dimensional cell culture matrices: state of the art, Tissue Eng. Part B Rev. 14 (2008) 61-86.

4. X. Le, G. Poinern, Gerrard E. Jai, N. Ali, C. M. Berry, D. Fawcett, Engineering a Biocompatible Scaffold with Either Micrometre or Nanometre Scale Surface Topography for Promoting Protein Adsorption and Cellular Response, Int. J. Biomater. (2013) e782549.

5. R. G. Flemming, C. J. Murphy, G. A. Abrams, S. L. Goodman, P. F. Nealey, Effects of synthetic micro- and nano-structured surfaces on cell behavior, Biomaterials 20 (1999) 573-588.

6. Y. Li, G. Huang, X. Zhang, L. Wang, Y. Du, T. J. Lu, F. Xu, Engineering cell alignment in vitro, Biotechnol. Adv. 32 (2014) 347-365.

7. W. Pfleging, M. Bruns, A. Welle, S. Wilson, Laser-assisted modification of polystyrene surfaces for cell culture applications, Appl. Surf. Sci. 253 (2007) 9177-9184.

8. R. Ortiz, S. Moreno-Flores, I. Quintana, M. Vivanco, J. R. Sarasua, J. L. Toca-Herrera, Ultra-fast laser microprocessing of medical polymers for cell engineering applications, Mater. Sci. Eng. C 37 (2014) 241-250.

9. J. Li, H. McNally, R. Shi, Enhanced neurite alignment on micro-patterned poly-L-lactic acid films, J. Biomed. Mater. Res. A 87A (2008) 392-404.

10 J. G. Fernandez, C. A. Mills, E. Martinez, M. J. Lopez-Bosque, X. Sisquella, A. Errachid, J. Samitier, Micro- and nanostructuring of freestanding, biodegradable, thin sheets of chitosan via soft lithography, J. Biomed. Mater. Res. A 85A (2008) 242-247.

11. J. S. Choi, Y. Piao, T. S. Seo, Generation of hierarchical nano- and microwrinkle structure for smooth muscle cell alignment, Biotechnol. Bioprocess Eng. 19 (2014) 269-275.

12. J. Zhang, J.-Y. Ou, N. Papasimakis, Y. Chen, K. F. MacDonald, N. I. Zheludev, Continuous metal plasmonic frequency selective surfaces, Opt. Express 19 (2011) 23279-23285.

13. M. D. Morariu, N. E. Voicu, E. Schäffer, Z. Lin, T. P. Russell, U. Steiner, Hierarchical structure formation and pattern replication induced by an electric field, Nat. Mater. 2 (2003) 48-52.

14. A. Määttänen, A. Fallarero, J. Kujala, P. Ihalainen, P. Vuorela, J. Peltonen, Printed paper-based arrays as substrates for biofilm formation, AMB Express 4 (2014) 1-12.

15. J. Sarfraz, A. Määttänen, P. Ihalainen, M. Keppeler, M. Lindén, J. Peltonen, Printed copper acetate based H2S sensor on paper substrate, Sens. Actuators B Chem. 173 (2014) 868-873.

16. M. Jin, X. Feng, J. Xi, J. Zhai, K. Cho, L. Feng, L. Jiang, Super-hydrophobic PDMS surface with ultra-low adhesive force, Macromol. Rapid Commun. 26 (2005) 1805-1809.

17. P. Ihalainen, A. Määttänen, M. Pesonen, P. Sjöberg, J. Sarfraz, R. Österbacka, J. Peltonen, Paper-supported nanostructured ultrathin gold film electrodes—Characterization and functionalization, Appl. Surf. Sci. 329 (2015) 321-329.

18. J. Sun, B. Bhushan, J. Tong, Structural coloration in nature, RSC Adv. 3 (2013) 14862-14889.

19. R. Leach, Toim., Characterisation of Areal Surface Texture. Berlin, Heidelberg: Springer Berlin Heidelberg, 2013.

20. X. Wang, G.-M. Ng, J.-W. Ho, H.-L. Tam, F. Zhu, Efficient Semitransparent Bulk-Heterojunction Organic Photovoltaic Cells With High-Performance Low Processing Temperature Indium #x2013; Tin Oxide Top Electrode, IEEE J. Sel. Top. Quantum Electron. 16 (2010) 1685-1689.

21. S. Norrman, T. Andersson, C. G. Granqvist, O. Hunderi, Optical properties of discontinuous gold films, Phys. Rev. B 18 (1978) 674-695.

22. P. Ihalainen, F. Pettersson, M. Pesonen, T. Viitala, A. Määttänen, R. Österbacka, J. Peltonen, An impedimetric study of DNA hybridization on paper-supported inkjet-printed gold electrodes, Nanotechnology 25 (2014) 094009.

23. P. Ihalainen, H. Majumdar, A. Määttänen, S. Wang, R. Österbacka, J. Peltonen, Versatile characterization of thiol-functionalized printed metal electrodes on flexible substrates for cheap diagnostic applications, Biochim. Biophys. Acta BBA 1830 (2013) 4391-4397.

24. P. Ihalainen, H. Majumdar, T. Viitala, B. Törngren, T. Närjeoja, A. Määttänen, J. Sarfraz, H. Härmä, M. Yliperttula, R. Österbacka, J. Peltonen, Application of Paper-Supported Printed Gold Electrodes for Impedimetric Immunosensor Development, Biosensors 3 (2012) 1-17.

25. C. Giacomelli, K. Ckless, D. Galato, F. S. Miranda, A. Spinelli, Electrochemistry of Caffeic Acid Aqueous Solutions with pH 2.0 to 8.5, J. Braz. Chem. Soc. 13 (2002) 332-338.

26. K. Asadpour-Zeynali, M. R. Majidi, M. Zarifi, Carbon ceramic electrode incorporated with zeolite ZSM-5 for determination of Piroxicam, Cent. Eur. J. Chem. 8 (2010) 155-162.

1. A transparent or semi-transparent latex film for flexible and semi-transparent electronics, wherein said film is self-supporting and comprises a nanostructured surface having a hierarchical morphology.

2. The film according to claim 1, wherein the film is transparent or semi-transparent, transmission of light in visible range being greater than 50%.

3. The film according to claim 1 or 2, wherein the film is transparent or semi-transparent, transmission of light in visible range being in the range of from 70% to 90% or from 70% to 99%.

4. The film according to any one of claims 1-3, wherein said film is producible with roll-to-roll processing.

5. The film according to any one of the previous claims, wherein said film comprises styrene and/or butadiene groups.

6. The film according to claim 5, wherein said film comprises a blend of two latexes.

7. The film according to claim 6, wherein said two latexes comprise polymers selected from the group consisting of: styrene, acrylonitrile, butadiene and copolymers thereof.

8. The film according to claim 6, wherein said two latexes are polystyrene and styrene butadiene acrylonitrile copolymer.

9. The film according to any one of the previous claims, wherein said film is in an assembly with a semi-transparent electrode.

10. The film according to claim 9, wherein said semi-transparent electrode is an ultrathin metal film electrode (UTMF) or a conductive semitransparent or transparent polymer such as PEDOT:PSS.

11. The film according to claim 9, wherein said semi-transparent electrode is an ultrathin gold film (UTGF) electrode.

12. The film according to any one of the previous claims, wherein said film is functionalized with a biomolecule, thiol monolayer, a semi-transparent electrode or a combination thereof.

13. A combination of a transparent or semi-transparent latex film for flexible and semi-transparent electronics and a transparent or non-transparent support, wherein said film is placed upon said support or said film is a coat applied to said support and wherein said film comprises a nanostructured surface having a hierarchical morphology.

14. The combination according to claim 13, wherein said film comprises styrene and/or butadiene groups.

15. The combination according to claim 14, wherein said film comprises a blend of two latexes.

16. The combination according to claim 15, wherein said two latexes comprise polymers selected from the group consisting of: styrene, acrylonitrile, butadiene and copolymers thereof.

17. The combination according to any one of claims 13-16, wherein said transparent support is made of glass or polymer material, e.g. a thermoplastic material, and the transmission of light in the visible range being greater than 50%.

18. The combination according to claim 17, wherein said polymer material is polydimethylsiloxane (PDMS), polyethylene terephthalate (PET), polyethylene naphthalate (PEN), or polylactic acid (PLA).

19. The combination according to any one of claims 13-16, wherein said non-transparent support comprises or is made of paper, plastic or cellulose fibers.

20. The combination according to any one of claims 13-19, wherein said film is functionalized with a biomolecule, thiol monolayer, a semi-transparent electrode or a combination thereof.

21. Semi-transparent assembly for monitoring and manipulating cellular processes in real-time, comprising a latex film according to any one of claims 1-10 and semi-transparent electrodes for high-resolution microscopy imaging and electrical monitoring in real-time.

22. The assembly according to claim 21, wherein said assembly is capable of providing detailed information on one of several of cell morphology, growth and migration for multi-wells.

23. The assembly according to claim 21 or 22, wherein said latex film is on a transparent support made of glass or polymer material, such as a thermoplastic material, with the transmission of light in visible range being over 50%.

24. The assembly according to claim 23 comprising a hierarchically structured latex formed by
a transparent modified glass coverslip substrate with a latex-based structured surface topography,
tailored surface chemistry and
semi-transparent electrodes.

25. The assembly according to any one of claims 21 to 24, wherein the materials are designed to control cell growth or induce cell migration in a desired manner.

26. The assembly according to any one of claims 21 to 25, wherein the electrodes allow for controlling cell status by electrical stimulus, or controlled drug release while simultaneously measuring pH and metabolites in the cell medium for real-time metabolic measurements.

27. The assembly according to any of claims 21 to 26, characterized in that it forms a well plate for in vitro cell culture studies.

28. Use of a paper supported functionalized latex-film for quantification of cell growth or activity on materials of interest, wherein said film comprises a nanostructured surface having a hierarchical morphology and said film is functionalized with a biomolecule, thiol monolayer, a semi-transparent electrode or a combination thereof.

29. The use according to claim 28, wherein said biomolecule is an active pharmaceutical ingredient (API).

30. Method for preparing a functionalized latex film, the method comprising the steps of:
a) coating a solid support with a latex blend comprising two polymers selected from the group consisting of: styrene, acrylonitrile, butadiene and copolymers thereof;
b) drying and sintering the latex film formed in step a);
c) optionally peeling the latex film from the support;
d) functionalizing the latex film obtained from step b) or c) by coating or printing on said film a biomolecule, thiol monolayer, a semi-transparent electrode or a combination thereof.

31. The method according to claim 30, wherein said latex blend is a blend of polystyrene and styrene butadiene acrylonitrile copolymer.
